SEQUENCE LISTING (8 sequences)

SEQ ID NO: 1 - DNA, 825 bp, Homo sapiens
atggtcggcagaagagcactgatcgtactggctcactcagagaggacgtccttcaactatgccatgaaggaggctgctgcagcggctttgaagaagaaaggatgggaggtggtggagtcggacctctatgccatgaacttcaatcccatcatttccagaaaggacatcacaggtaaactgaaggaccctgcgaactttcagtatcctgccgagtctgttctggcttataaagaaggccatctgagcccagatattgtggctgaacaaaagaagctggaagccgcagaccttgtgatattccagttccccctgcagtggtttggagtccctgccattctgaaaggctggtttgagcgagtgttcataggagagtttgcttacacttacgctgccatgtatgacaaaggacccttccggagtaagaaggcagtgctttccatcaccactggtggcagtggctccatgtactctctgcaagggatccacggggacatgaatgtcattctctggccaattcagagtggcattctgcatttctgtggcttccaagtcttagaacctcaactgacatatagcattgggcacactccagcagacgcccgaattcaaatcctggaaggatggaagaaacgcctggagaatatttgggatgagacaccactgtattttgctccaagcagcctctttgacctaaacttccaggcaggattcttaatgaaaaaagaggtacaggatgaggagaaaaacaagaaatttggcctttctgtgggccatcacttgggcaagtccatcccaactgacaaccagatcaaagctagaaaatga

SEQ ID NO: 2 - protein, 274 aa, Homo sapiens
MVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQFPLQWFGVPAILKGWFERVFIGEFAYTYAAMYDKGPFRSKKAVLSITTGGSGSMYSLQGIHGDMNVILWPIQSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARK

SEQ ID NO: 3 - DNA, 825 bp, Homo sapiens; identical to SEQ ID NO: 1

SEQ ID NO: 4 - protein, 274 aa, Homo sapiens; identical to SEQ ID NO: 2

SEQ ID NO: 5 - DNA, 723 bp, Homo sapiens
atggtcggcagaagagcactgatcgtactggctcactcagagaggacgtccttcaactatgccatgaaggaggctgctgcagcggctttgaagaagaaaggatgggaggtggtggagtcggacctctatgccatgaacttcaatcccatcatttccagaaaggacatcacaggtaaactgaaggaccctgcgaactttcagtatcctgccgagtctgttctggcttataaagaaggccatctgagcccagatattgtggctgaacaaaagaagctggaagccgcagaccttgtgatattccagttccccctgcagtggtttggagtccctgccattctgaaaggctggtttgagcgagtgttcataggagagtttgcttacacttacgctgccatgtatgacaaaggacccttccggagtggcattctgcatttctgtggcttccaagtcttagaacctcaactgacatatagcattgggcacactccagcagacgcccgaattcaaatcctggaaggatggaagaaacgcctggagaatatttgggatgagacaccactgtattttgctccaagcagcctctttgacctaaacttccaggcaggattcttaatgaaaaaagaggtacaggatgaggagaaaaacaagaaatttggcctttctgtgggccatcacttgggcaagtccatcccaactgacaaccagatcaaagctagaaaatga

SEQ ID NO: 6 - protein, 240 aa, Homo sapiens
MVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQFPLQWFGVPAILKGWFERVFIGEFAYTYAAMYDKGPFRSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARK

SEQ ID NO: 7 - DNA, 711 bp, Homo sapiens
atggtcggcagaagagcactgatcgtactggctcactcagagaggacgtccttcaactatgccatgaaggaggctgctgcagcggctttgaagaagaaaggatgggaggtggtggagtcggacctctatgccatgaacttcaatcccatcatttccagaaaggacatcacaggtaaactgaaggaccctgcgaactttcagtatcctgccgagtctgttctggcttataaagaaggccatctgagcccagatattgtggctgaacaaaagaagctggaagccgcagaccttgtgatattccagagtaagaaggcagtgctttccatcaccactggtggcagtggctccatgtactctctgcaagggatccacggggacatgaatgtcattctctggccaattcagagtggcattctgcatttctgtggcttccaagtcttagaacctcaactgacatatagcattgggcacactccagcagacgcccgaattcaaatcctggaaggatggaagaaacgcctggagaatatttgggatgagacaccactgtattttgctccaagcagcctctttgacctaaacttccaggcaggattcttaatgaaaaaagaggtacaggatgaggagaaaaacaagaaatttggcctttctgtgggccatcacttgggcaagtccatcccaactgacaaccagatcaaagctagaaaatga

SEQ ID NO: 8 - protein, 236 aa, Homo sapiens
MVGRRALIVLAHSERTSFNYAMKEAAAAALKKKGWEVVESDLYAMNFNPIISRKDITGKLKDPANFQYPAESVLAYKEGHLSPDIVAEQKKLEAADLVIFQSKKAVLSITTGGSGSMYSLQGIHGDMNVILWPIQSGILHFCGFQVLEPQLTYSIGHTPADARIQILEGWKKRLENIWDETPLYFAPSSLFDLNFQAGFLMKKEVQDEEKNKKFGLSVGHHLGKSIPTDNQIKARK

(19) United States
(12) Patent Application Publication
(10) Pub. No.: US 2014/0155361 A1
(43) Pub. Date: Jun. 5, 2014

(54) METHOD FOR TREATING LUNG DISEASE

(71) Applicant: Duke University, Durham, NC (US)

(72) Inventors: John W. Hollingsworth, Durham, NC (US); William Michael Foster, Durham, NC (US); Erin Potts-Kant, Chapel Hill, NC (US); Zhuowei Li, Chapel Hill, NC (US)

(21) Appl. No.: 14/175,155

(22) Filed: Feb. 7, 2014

Related U.S. Application Data
(63) Continuation of application No. 13/313,832, filed on Dec. 7, 2011.
(60) Provisional application No. 61/481,225, filed on May 1, 2011.
(60) Provisional application No. 61/478,923, filed on Apr. 25, 2011.
(60) Provisional application No. 61/420,540, filed on Dec. 7, 2010.

(51) Int. Cl. A61K 31/352; A61K 45/06; A61K 31/343

(52) U.S. Cl. CPC A61K 31/352; A61K 31/343; A61K 45/06
USPC 514/171; 514/454; 514/468; 435/26

(57) ABSTRACT

Methods of treating lung diseases comprising administering inducers of NAD(P)H:quinone oxidoreductase 1 (NQO1) are disclosed. Inducers of NQO1 include naphthoquinones such as β-lapachone. Methods of predicting whether a subject with a lung disease will respond to treatment with a naphthoquinone are also described herein.

### CROSS-REFERENCE TO RELATED APPLICATIONS

This application claims priority to U.S. Provisional Application No. 61/420,540, filed on Dec. 7, 2010, U.S. Provisional Application No. 61/478,923, filed on Apr. 25, 2011, and U.S. Provisional Application No. 61/481,225, filed on May 1, 2011, the contents of each of which are hereby incorporated by reference.

### STATEMENT REGARDING FEDERALLY SPONSORED RESEARCH

This invention was made with United States Government support under Grant Nos. R01 ES016126 and ES016347, both awarded by the National Institutes of Health. The government has certain rights in the invention.

### SEQUENCE LISTING

The sequence listing is filed with the application in electronic format only and is incorporated by reference herein. The sequence listing text file “028193-9097-US03_ST25.txt” was created on Dec. 7, 2011, and is 13,792 bytes in size.

### INTRODUCTION

Chronic obstructive pulmonary disease (COPD) is the fourth-leading cause of death in the world and is the only disease in the top ten causes of death with increasing rates of mortality. COPD is characterized by irreversible defects in air flow and progressive loss of lung function. The development of pulmonary emphysema is a frequent observation in patients with COPD. Tobacco smoke is a dominant risk factor for the development of emphysema, however only 15-20% of smokers develop clinically recognized emphysema and approximately 25% of patients with emphysema are lifelong non-smokers. Accordingly, host factors may contribute to disease susceptibility.

Current therapies are focused on symptomatic treatment with bronchodilators, such as anti-cholinergics or beta-agonists, or inhaled steroids. However, there are currently no effective treatments for the progressive loss of lung function in COPD.

### SUMMARY

In one aspect, the disclosure may provide a method of treating a lung disease in a subject, comprising administering to the subject an effective amount of a naphthoquinone.

In another aspect, the disclosure may provide a method of treating lung disease in a subject, comprising administering to the subject an effective amount of an inducer of NQO1.

In another aspect, the disclosure may provide a method of treating a lung disease in a subject, comprising determining the level of NQO1 in a subject suffering from lung disease, and if the level of NQO1 is lower than a standard level, administering an effective amount of an inducer of NQO1.

In another aspect, the invention may provide a method of predicting responsiveness of a subject with a lung disease to treatment with a naphthoquinone, comprising:

a) detecting the level or activity of NQO1 in a sample obtained from the subject; and

b) comparing the level or activity of NQO1 in the sample to a standard level, wherein an altered level or activity of NQO1 indicates that the subject is responsive to treatment with a naphthoquinone.

In another aspect, the invention may provide a method of identifying a subject with a lung disease as a candidate for treatment with a naphthoquinone, comprising:

a) detecting the level or activity of NQO1 in a sample obtained from the subject; and

b) comparing the level or activity of NQO1 in the sample to a standard level, wherein a difference in the level or activity of NQO1 indicates that the subject is a candidate for treatment with a naphthoquinone.

In a further aspect, the invention may provide a method of identifying a subject having an increased risk of developing a lung disease comprising:

a) determining the presence or absence of a functional variant of NQO1 in the sample; and

b) identifying the patient as having an increased risk of developing lung disease when the sample a functional variant of NQO1 is present in the sample.

Other aspects and embodiments will become apparent in light of the following disclosure and drawings.

### BRIEF DESCRIPTION OF THE DRAWINGS

FIG. 1 illustrates the pulmonary effect of NQO1 deficiency in mice. (A) Gross lung photographs of lungs from NQO1 deficient mice compared to those from C57BL/6 mice at varying ages. (B) Lung histologies NQO1 deficient mice compared to those from C57BL/6 mice at varying ages. (C)-(E) mean line intercept (MLI) (C), static compliance (D), and residual volume (E) of NQO1 deficient mice compared to C57BL/6 mice.

FIG. 2 illustrates lung histologies (A) and lavage return volumes (B) for naive age-matched C57B/6 and NQO1−/− mice, evaluated at 1, 2, 4 and 6 months. The values are presented as the mean±SEM (*P<0.05).

FIG. 3 illustrates pressure volume curves of NQO1 deficient mice compared to C57BL/6 mice at one (A), two (B), four (C) and six (D) months of age. Ppl is the airway tracheal pressure measured at each given volume (Vpl) on inflation and deflation steps (n=10 with 2 repeats).

FIG. 4 illustrates the of effect elastase treatment in NQO1 deficient mice compared to C57BL/6 mice. (A) Static lung compliance. (B) Mean line intercept (MLI). (C) and (D) Pressure volume measurements in the presence or absence of lipopolysaccharide (LPS) and N-acetylcysteine (NAC). (E) 8-isoprostane and protein carbonyl concentrations (measurements of oxidative stress) were quantitated from the BALF. The values in A, B and E represent the mean SEM of evaluations of >10 mice with 2 repeats (*P<0.05).

FIG. 5 illustrates the effect of elastase treatment in NQO1 deficient mice compared to C57BL/6 mice, with or without treatment with N-acetylcysteine (NAC). (A) Alveolar surface density in NQO1−/− and WT mice. (B) Lavage returns after filling lung to total lung capacity three times. (C) Glutathione (GSH) measurements. (D) Total cell counts. (E) Macrophage counts. (F) Neutrophil counts. The alveolar surface density measurements, average lavage return volume, GSH measurements and cell counts were performed on 10 mice per group with 2 repeats. The values are presented as the mean SEM (*P<0.05).

FIG. 6 illustrates the effect of oxidant stress in NQO1 deficient mice compared to C57BL/6 mice. (A) Static lung compliance. (B) Alveolar space enlargement (MLI). (C) Pressure volume measurements. (D) Pressure volume measurements in the presence of LPS. (E) 8-isoprostane and protein carbonyl levels in bronchoalveolar lavage fluid (BALF).

FIG. 7 illustrates the effect of oxidant stress in NQO1 deficient mice compared to C57BL/6 mice. (A) Alveolar surface density. (B) Average lavage returns after filling lungs to total capacity three times. (C) Glutathione (GSH) measurements. (D) Total cell counts. (E) Macrophage counts. (F) Neutrophil counts. The alveolar surface density measurements, average lavage return volume, GSH measurements and cell counts were performed on 10 mice per group with 2 repeats. The values are presented as the mean SEM (*P<0.05).

FIG. 8 illustrates macrophage derived oxidant stress in NQO1 deficient mice compared to C57BL/6 mice. Alveolar macrophages from BAL were obtained, cultured, and stimulated with saline, LPS or phorbol myristate acetate (PMA). (A) 8-isoprostane and protein carbonyl concentrations. (B) 8-isoprostane and protein carbonyl concentrations after pre-treatment with NQO1 inhibitor MAC220. (C) 8-isoprostane and protein carbonyl concentrations after pre-treatment with NQO1 inhibitor dicumarol. (D) 8-isoprostane and protein carbonyl concentrations after pre-treatment with NQO1 inducer β-lapachone.

FIG. 9 illustrates MMP12 expression in NQO1 deficient mice compared to C57BL/6 mice.

FIG. 10 illustrates DLCO measurements for individuals genotyped for a common functional variant of NQO1 (NQO1 Pro187Ser: rs 1800566).

### DETAILED DESCRIPTION

Before any embodiments of the invention are explained in detail, it is to be understood that the invention is not limited in its application to the details of construction and the arrangement of components set forth in the following description or illustrated in the following drawings. The invention is capable of other embodiments and of being practiced or of being carried out in various ways. Also, it is to be understood that the phraseology and terminology used herein is for the purpose of description and should not be regarded as limiting. The use of “including,” “comprising,” or “having” and variations thereof herein is meant to encompass the items listed thereafter and equivalents thereof as well as additional items.

### Definitions

“Administration” or “administering” refers to providing, contacting, and/or delivery of a compound or compounds by any appropriate route to achieve the desired effect. Administration may include, but is not limited to, oral, sublingual, intramuscular, subcutaneous, intravenous, transdermal, topical, parenteral, buccal, rectal, and via injection, inhalation, and implants.

The term “contacting” when used as in “contacting a cell” refers to contacting a cell directly or indirectly in vitro, ex vivo, or in vivo (i.e. within a subject, such as a mammal, including humans, mice, rats, rabbits, cats, and dogs). Contacting a cell, which also includes “reacting” a cell, can occur as a result of administration to a subject. Contacting encompasses administration to a cell, tissue, mammal, subject, patient, or human. Further, contacting a cell includes adding an agent to a cell culture. Other suitable methods may include introducing or administering an agent to a cell, tissue, mammal, subject, or patient using appropriate procedures and routes of administration as defined above.

The term “effective amount” refers to a dosage of a compound or compounds or compositions effective for eliciting a desired effect. This term as used herein may also refer to an amount effective at bringing about a desired in vivo effect such as, for example, in an animal, including in a human.

The term “inducer” when used herein as in “inducer of NQO1” refers to an agent that increases (e.g., measurably increases) the activity of NQO1, the amount of NQO1, or the expression level of NQO1, or causes NQO1 activity to increase to a level that is greater than a typical basal NQO1 level of activity. The inducer can increase the activity of NQO1 either directly or indirectly. The inducer may be any suitable agent such as, for example, a small molecule or a biomolecule (e.g., a protein, peptide or a nucleic acid). An agent can be evaluated to determine if it is an inducer by measuring either directly or indirectly the activity of the NQO1 when subjected to the agent. The activity of the agent can be measured, for example, against a control substance.

The term “sample,” as used herein in the context of a sample from a subject, refers to a any biological specimen obtained from a subject. For example, the sample may be a cell, a body fluid or a tissue from the subject.

The term “treatment,” as used herein in the context of treating a condition, pertains generally to treatment and therapy, whether of a human or an animal (e.g., in veterinary applications), in which a desired therapeutic effect is achieved. For example, treatment includes prophylaxis and may ameliorate or remedy the condition, disease, or symptom, or may inhibit the progress of the condition, symptom, or disease (e.g., reduce the rate of progress or halt the rate of progress).

### NAD(P)H:Quinone Oxidoreductase 1 (NQO1)

NAD(P)H:quinone oxidoreductase 1 (NQO1) is a member of the NAD(P)H dehydrogenase (quinone) family and encodes a cytoplasmic 2-electron reductase. It is an FAD-binding protein which reduces quinones to hydroquinones. The protein's enzymatic activity prevents the one electron reduction of quinones that results in the production of radical species, and NQO1 may act as an antioxidant enzyme by regenerating antioxidant forms of ubiquinone and vitamin E quinone. Mutations in the gene have been associated with tardive dyskinesia (TD), an increased risk of hematotoxicity after exposure to benzene, and susceptibility to various forms of cancer. Altered expression of this protein has been observed in many tumors and is also associated with Alzheimer's disease (AD).

As used herein “NQO1” can relate to any mammalian (rat, mouse, human, etc.) NQO1 polynucleotide (DNA, cDNA, RNA, mRNA, etc.) or polypeptide (protein, peptide or fragment thereof, etc.) sequence as well as transcriptional and splice variants thereof. Non-limiting examples of NQO1 sequences include those having GenBank accession numbers BC007659, NM_{—}000903, NM_{—}001025433, and NM_{—}001025434 (see, e.g., SEQ ID NOs 1-8).

Previous work support that activation of nuclear factor erythroid 2-related factor 2 (Nrf2) is protective to the lung through induction of hundreds of antioxidant genes. In models of lung injury, the expression of NQO1 is upregulated in a manner dependent on Nrf2 and human emphysema is associated with reduced levels of NQO1. However, the functional role of NQO1 in emphysema remains unknown.

Without being limited by any particular theory, the inventors have found that NQO1 may play an active role in the development of emphysema. Using a mouse model, it has been identified that NQO1−/− mice develop premature senile emphysema (histology and physiology). NQO1−/− mice are susceptible in both an elastase model of emphysema and LPS-induced emphysema. Furthermore, NQO1−/− is associated with increased oxidant stress in each of the above challenges. Antioxidants have also been found to reverse the enhanced emphysema associated with lipopolysaccharide (LPS) exposure in NQO1−/− animals.

Further, in vitro studies show that macrophages are a source of NQO1-dependent oxidative stress. Oxidative stress is associated with progression or exacerbation of many lung diseases. Upon stimulation, NQO1−/− macrophages generate increased levels of MMP12. The role of NQO1 in stress-induced MMP12 expression is further supported by in vitro studies utilizing chemical induction of NQO1 in macrophages, which attenuate both oxidative stress and expression of MMP12. The role of MMP12 in human airway disease and COPD has recently been reported. (Hunninghake et al. New Engl. J. Med. 361:2599-2608 (2009)).

Therefore, it may be possible to treat COPD by enhancing or activating NQO1.

### Naphthoquinones

A naphthoquinone is an organic compound C10H6O2. It can be viewed as a derivative of naphthalene with two hydrogen atoms replaced by two ketone moieties. Three common isomers of naphthoquinones are 1,2-naphthoquinone, 1,4-naphthoquinone and 2,6-naphthoquinone.

β-lapachone (3,4-dihydro-2,2-dimethyl-2H-naphtho[1,2-b]pyran-5,6-dione), is a 1,2-naphthoquione derived from lapachol, a 1,4-naphthoquinone). β-lapachone can be isolated from the lapacho tree (Tabebuia avellanedae), a member of the catalpa family (Bignoniaceae). Lapachol and β-lapachone (with numbering) have the following chemical structures:

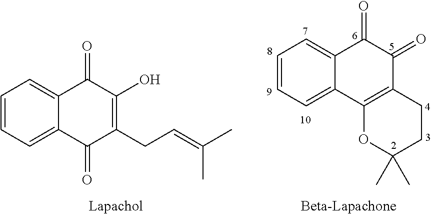 [SMILES: CC(C)=CCC1=C(O)C(=O)c2ccccc2C1=O ; CC1(C)CCC2=C(O1)c1ccccc1C(=O)C2=O]

β-lapachone, as well as its intermediates, derivatives and analogs thereof, are described in Li et al. (1993) J. Biol. Chem., 268(30): 22463-22468. As a single agent, β-lapachone has demonstrated significant antineoplastic activity against human cancer cell lines at concentrations typically in the range of 1-10 μM (IC50). β-lapachone may induce unscheduled expression of checkpoint molecules. Although its exact intracellular target(s) and mechanism of antineoplastic activity remain unknown, β-lapachone has also shown potent in vitro inhibition of human DNA Topoisomerases I and II (Li et al. (1993) J. Biol. Chem. 268: 22463; and Frydman et al. (1997) Cancer Res. 57: 620) with novel mechanisms of action. Unlike topoisomerase “poisons” (e.g., camptothecin, etoposide, doxorubicin) which stabilize the covalent topoisomerase-DNA complex and induce topoisomerase-mediated DNA cleavage, β-lapachone interacts either directly with the enzyme to inhibit catalysis and block the formation of cleavable complex (Li et al. (1993) J. Biol. Chem. 268: 22463) or with the complex itself, causing religation of DNA breaks and dissociation of the enzyme from DNA (Krishnan et al. (2000) Biochem. Pharm., 60: 1367).

Another possible intracellular target for β-lapachone in tumor cells is NQO1. Biochemical studies suggest that reduction of β-lapachone by NQO1 leads to a “futile cycling” between the quinone and hydroquinone forms with a concomitant loss of reduced NADH or NAD(P)H (Pink et al. (2000) J. Biol Chem. 275: 5416). The exhaustion of these reduced enzyme cofactors may be a critical factor for the activation of the apoptotic pathway after β-lapachone treatment.

A number of β-lapachone analogs have been disclosed in the art, such as those described in PCT International Application PCT/US93/07878 (WO94/04145) and U.S. Pat. No. 6,245,807, in which a variety of substituents may be attached at positions 3- and 4- on the β-lapachone compound. PCT International Application PCT/US00/10169 (WO 00/61142), discloses β-lapachone analogs, which may have a variety of substituents at the 3-position as well as in place of the methyl groups attached at the 2-position. U.S. Pat. Nos. 5,763,625, 5,824,700, and 5,969,163, disclose analogs and derivatives with a variety of substituents at the 2-, 3- and 4-positions. Furthermore, a number of journals report β-lapachone analogs and derivatives with substituents at one or more of the following positions: 2-, 3-, 8- and/or 9-positions, (See, Sabba et al. (1984) J. Med. Chem. 27:990-994 (substituents at the 2-, 8- and 9-positions); (Portela and Stoppani, (1996) Biochem. Pharm. 51:275-283 (substituents at the 2- and 9-positions); Goncalves et al. (1998) Mol. Biochem. Parasit. 1:167-176 (substituents at the 2- and 3-positions)). Moreover, U.S. Patent Application Publication No. 2004/0266857 and PCT International Application PCT/US2003/037219 (WO 04/045557), disclose structures having sulfur-containing hetero-rings in the “α” and “β” positions of lapachone (Kurokawa (1970) B. Chem. Soc. Jpn. 43:1454-1459; Tapia et al. (2000) Heterocycles 53(3):585-598; Tapia et al. (1997) Tetrahedron Lett. 38(1):153-154; Chuang et al. (1996) Heterocycles 40(10):2215-2221; Suginome et al. (1993) J. Chem. Soc. Chem. Comm. 9: 807-809; Tonholo et al. (1988) J. Brazil. Chem. Soc. 9(2): 163-169; and Krapcho et al. (1990) J. Med. Chem. 33(9):2651-2655).

Other 1,2-naphthoquinones that may be used in the methods described herein include tanshinone IIA (1,6,6-trimethyl-8,9-dihydro-7H-naphtho[1,2-g][1]benzofuran-10,11-dione) and cryptotanshinone ((R)-1,2,6,7,8,9-Hexahydro-1,6,6-trimethyl-phenanthro(1,2-b)furan-10,11-dione).

### Other Inducers of NQO1

There are other chemical compounds that can induce the level or activity of NQO1, and any of these compounds may also be used in accordance with the disclosure. These compounds include:

(1) Extracts of Brassica (broccoli) (Zhang et al. (2006) J. Agric. Food. Chem. 54:9370-9376)
i. ascorbigen, a natural compound derived from glucosinolates (Wagner et al. (2008) Ann. Nutr. Metab. 53:122-128)
ii. isothiocyanate sulforaphane (Dinkova-Kostova et al. (2007) Cancer Epidemiol. Biomarkers Prev. 16: 847-851)
iii. sulforaphane (Dinkova-Kostova et al. (2007) Cancer Epidemiol. Biomarkers Prev. 16: 847-851)

(2) Inducers of dopamine including bromocriptine (Jia et al. (2008) Neurochem. Res. 33: 2197-2205; Lim et al. (2008) Pharmacol. Res. 57: 325-331)

(3) oleanane dicyanotriterpenoid 2-cyano-3,12-dioxooleana-1,9(11)-dien-28-onitrile (TP-225) (Dinkova-Kostova et al. (2008) Biochem. Biophys. Res. Commun. 367: 859-865)

(4) synthetic triterpenoid (TP) analogues of oleanolic acid (Dinkova-Kostova et al. (2005) Proc. Natl. Acad. Sci. U.S.A. 102: 4584-4589)

(5) dimethyl fumarate (DMF) (Digby et al. (2005) Cancer Chemother. Pharmacol. 56: 307-316; Begleiter et al. (2004) Br. J. Cancer 91: 1624-1631)

(6) oxathiolene oxides (OTEOs) (Pietsch et al. (2003) Biochem. Pharmacol. 65: 1261-1269)

(7) dithiolethiones such as oltipraz, including 3H-1,2-dithiole-3-thione (D3T) (Kwak et al. (2001) Mol. Med. 7:135-145; Begleiter et al. (2003) Cancer Epidemiol. Biomarkers Prev. 12: 566-572)

(8) Caffeic acid phenethyl ester (CAPE) (Jaiswal et al. (1997) Cancer Res. 57: 440-446)

(9) β-naphthoflavone (BNF)

(10) ellipticine

(11) Xenobiotics (e.g., β-naphthoflavone)

(12) Antioxidants (e.g., 2(3)-tert-butyl-4-hydroxyanisole (BHA) and tert-butylhydroquinone (tBHQ)

(13) Sulforaphane

(14) p-tyrosol

### Lung Diseases

Described herein are methods for treating lung diseases. Exemplary lung diseases include, but are not limited to, COPD, chronic bronchitis, emphysema, asthma, pulmonary fibrosis and reactive airway disease.

COPD is a term which refers to a large group of lung diseases which can interfere with normal breathing. Current clinical guidelines define COPD as a disease state characterized by airflow limitation that is not fully reversible. The airflow limitation is usually both progressive and associated with an abnormal inflammatory response of the lungs to noxious particles and gases. The most relevant contributory source of such particles and gases, at least in the western world, is tobacco smoke. COPD patients can exhibit a variety of symptoms, including cough, shortness of breath, and excessive production of sputum; such symptoms arise from dysfunction of a number of cellular compartments, including neutrophils, macrophages, and epithelial cells. The two most clinically relevant conditions covered by COPD are chronic bronchitis and emphysema.

Chronic bronchitis is a long-standing inflammation of the bronchi which causes increased production of mucous and other changes. The patients' typical symptoms are cough and expectoration of sputum. Chronic bronchitis can lead to more frequent and severe respiratory infections, narrowing and plugging of the bronchi, difficult breathing and disability.

Emphysema is a chronic lung disease which affects the alveoli and/or the ends of the smallest bronchi. The lung loses its elasticity and therefore these areas of the lungs become enlarged. These enlarged areas trap stale air and do not effectively exchange it with fresh air. This results in difficult breathing and may result in insufficient oxygen being delivered to the blood. The predominant symptom in patients with emphysema is shortness of breath. The development of pulmonary emphysema is a frequent observation in patients with COPD. The incidence of emphysema is reaching worldwide epidemic proportions and predicted to displace stroke as the third major worldwide cause of mortality by 2030. The pathologic feature of pulmonary emphysema is alveolar destruction with the loss of lung functional units. The development of emphysema is accompanied by accumulation of inflammatory cells such as macrophages and neutrophils in the airways and lung parenchyma. The molecular pathogenesis of emphysema includes both protease-antiprotease imbalance and oxidant stress.

Asthma is generally defined as an inflammatory disorder of the airways with clinical symptoms arising from intermittent airflow obstruction. It is characterized clinically by paroxysms of wheezing, dyspnea and cough. It is a chronic disabling disorder that appears to be increasing in prevalence and severity. It is estimated that 15% of children and 5% of adults in the population of developed countries suffer from asthma. Therapy should therefore be aimed at controlling symptoms so that normal life is possible and at the same time provide basis for treating the underlying inflammation.

Pulmonary fibrosis is the formation or development of excess fibrous connective tissue (fibrosis) in the lungs. It can be described as “scarring of the lung”. Pulmonary fibrosis involves gradual replacement of normal lung parenchyma with fibrotic tissue. Thickening of scar tissue causes irreversible decrease in oxygen diffusion capacity. In addition, decreased compliance makes pulmonary fibrosis a restrictive lung disease. It is the main cause of restrictive lung disease that is intrinsic to the lung parenchyma.

Reactive airway disease is a general term for conditions involving wheezing and allergic reactions. It is sometimes mistakenly used as a synonym for asthma. It is sometimes used to refer to an asthma-like syndrome often developed after a single exposure to high levels of a trigger, such as irritating vapor, fume, or smoke. Another current usage of the term in the medical community is to describe an asthma-like syndrome in infants that may later be confirmed to be asthmatics when they become old enough to participate in diagnostic tests.

### Formulations

While the naphthoquinone may be administered alone in the methods described herein, it may also be presented as a pharmaceutical composition (e.g., a formulation) comprising at least a naphthoquinone, as described above, together with one or more pharmaceutically acceptable carriers, adjuvants, excipients, diluents, fillers, buffers, stabilizers, preservatives, lubricants, or other materials well known to those skilled in the art and optionally other therapeutic or prophylactic agents.

Thus, the methods described herein include administration of a pharmaceutical composition, as defined above, in which a naphthoquinone is admixed together with one or more pharmaceutically acceptable carriers, excipients, buffers, adjuvants, stabilizers, or other materials, as described herein.

Suitable carriers, excipients, etc. can be found in standard pharmaceutical texts, for example, Remington's Pharmaceutical Sciences, 18th edition, Mack Publishing Company, Easton, Pa., 1990.

The formulations may conveniently be presented in unit dosage form and may be prepared by any methods well known in the art of pharmacy. Such methods include the step of bringing into association the active compound with the carrier which constitutes one or more accessory ingredients. In general, the formulations are prepared by uniformly and intimately bringing into association the active compound with liquid carriers or finely divided solid carriers or both, and then if necessary shaping the product.

Formulations may be in the form of liquids, solutions, suspensions, emulsions, elixirs, syrups, tablets, lozenges, granules, powders, capsules, cachets, pills, ampoules, suppositories, pessaries, ointments, gels, pastes, creams, sprays, mists, foams, lotions, oils, boluses, electuaries, or aerosols.

Formulations suitable for oral administration (e.g. by ingestion) may be presented as discrete units such as capsules, cachets or tablets, each containing a predetermined amount of the active compound; as a powder or granules; as a solution or suspension in an aqueous or non-aqueous liquid; or as an oil-in-water liquid emulsion or a water-in-oil liquid emulsion; as a bolus; as an electuary; or as a paste.

A tablet may be made by conventional means, e.g., compression or molding, optionally with one or more accessory ingredients. Compressed tablets may be prepared by compressing in a suitable machine the active compound in a free-flowing form such as a powder or granules, optionally mixed with one or more binders (e.g. povidone, gelatin, acacia, sorbitol, tragacanth, hydroxypropylmethyl cellulose); fillers or diluents (e.g. lactose, microcrystalline cellulose, calcium hydrogen phosphate); lubricants (e.g. magnesium stearate, talc, silica); disintegrants (e.g. sodium starch glycolate, cross-linked povidone, cross-linked sodium carboxymethyl cellulose); surface-active or dispersing or wetting agents (e.g. sodium lauryl sulfate); and preservatives (e.g. methyl p-hydroxybenzoate, propyl p-hydroxybenzoate, sorbic acid). Molded tablets may be made by molding in a suitable machine a mixture of the powdered compound moistened with an inert liquid diluent. The tablets may optionally be coated or scored and may be formulated so as to provide slow or controlled release of the active compound therein using, for example, hydroxypropylmethyl cellulose in varying proportions to provide the desired release profile. Tablets may optionally be provided with an enteric coating, to provide release in parts of the gut other than the stomach.

Formulations suitable for topical administration (e.g. transdermal, intranasal, ocular, buccal, and sublingual) may be formulated as an ointment, cream, suspension, lotion, powder, solution, past, gel, spray, aerosol, or oil. Alternatively, a formulation may comprise a patch or a dressing such as a bandage or adhesive plaster impregnated with active compounds and optionally one or more excipients or diluents.

Formulations suitable for topical administration in the mouth include lozenges comprising the active compound in a flavored basis, usually sucrose and acacia or tragacanth; pastilles comprising the active compound in an inert basis such as gelatin and glycerin, or sucrose and acacia; and mouthwashes comprising the active compound in a suitable liquid carrier.

Formulations suitable for topical administration to the eye also include eye drops wherein the active compound is dissolved or suspended in a suitable carrier, especially an aqueous solvent for the active compound.

Formulations suitable for nasal administration, wherein the carrier is a solid, include a coarse powder having a particle size, for example, in the range of about 20 to about 500 microns which is administered in the manner in which snuff is taken, i.e. by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the active compound.

Formulations suitable for administration by inhalation include those presented as an aerosol spray from a pressurized pack, with the use of a suitable propellant, such as dichlorodifluoromethane, trichlorofluoromethane, dichoro-tetrafluoroethane, carbon dioxide, or other suitable gases. Further formulations suitable for inhalation include those presented as a nebulizer.

Formulations suitable for topical administration via the skin include ointments, creams, and emulsions. When formulated in an ointment, the active compound may optionally be employed with either a paraffinic or a water-miscible ointment base. Alternatively, the active compounds may be formulated in a cream with an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the active compound through the skin or other affected areas. Examples of such dermal penetration enhancers include dimethylsulfoxide and related analogues.

When formulated as a topical emulsion, the oily phase may optionally comprise merely an emulsifier (otherwise known as an emulgent), or it may comprises a mixture of at least one emulsifier with a fat or an oil or with both a fat and an oil. Preferably, a hydrophilic emulsifier is included together with a lipophilic emulsifier which acts as a stabilizer. It is also preferred to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Suitable emulgents and emulsion stabilizers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the active compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as diisoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for rectal administration may be presented as a suppository with a suitable base comprising, for example, cocoa butter or a salicylate.

Formulations suitable for vaginal administration may be presented as pessaries, tampons, creams, gels, pastes, foams or spray formulations containing in addition to the active compound, such carriers as are known in the art to be appropriate.

Formulations suitable for parenteral administration (e.g. by injection, including cutaneous, subcutaneous, intramuscular, intravenous and intradermal), include aqueous and nonaqueous isotonic, pyrogen-free, sterile injection solutions which may contain anti-oxidants, buffers, preservatives, stabilizers, bacteriostats, and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. Examples of suitable isotonic vehicles for use in such formulations include Sodium Chloride Injection, Ringer's Solution, or Lactated Ringer's Injection. Typically, the concentration of the active compound in the solution is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules, and tablets. Formulations may be in the form of liposomes or other microparticulate systems which are designed to target the active compound to blood components or one or more organs.

### Dosages

It will be appreciated that appropriate dosages of the active compounds, and compositions comprising the active compounds, can vary from patient to patient. Determining the optimal dosage will generally involve the balancing of the level of therapeutic benefit against any risk or deleterious side effects of the treatments of the present invention. The selected dosage level will depend on a variety of factors including, but not limited to, the activity of the particular compound, the route of administration, the time of administration, the rate of excretion of the compound, the duration of the treatment, other drugs, compounds, and/or materials used in combination, and the age, sex, weight, condition, general health, and prior medical history of the patient. The amount of compound and route of administration will ultimately be at the discretion of the physician, although generally the dosage will be to achieve local concentrations at the site of action which achieve the desired effect without causing substantial harmful or deleterious side-effects.

Administration in vivo can be effected in one dose, continuously or intermittently (e.g. in divided doses at appropriate intervals) throughout the course of treatment. Methods of determining the most effective means and dosage of administration are well known to those of skill in the art and will vary with the formulation used for therapy, the purpose of the therapy, the target cell being treated, and the subject being treated. Single or multiple administrations can be carried out with the dose level and pattern being selected by the treating physician.

In general, a suitable dose of the active compound is in the range of about 100 μg to about 250 mg per kilogram body weight of the subject per day. Where the active compound is a salt, an ester, prodrug, or the like, the amount administered is calculated on the basis of the parent compound and so the actual weight to be used is increased proportionately.

### Combination Therapies

In some embodiments, an additional active agent or agents can be administered with a naphthoquinone in the methods of the present invention. The additional active agent or agents can be administered simultaneously or sequentially with the naphthoquinone. Sequential administration includes administration before or after the naphthoquinone. In some embodiments, the additional active agent or agents can be administered in the same composition as the naphthoquinone. In other embodiments, there can be an interval of time between administration of the additional active agent and the naphthoquinone. In some embodiments, the administration of an additional therapeutic agent with naphthoquinone will enable lower doses of the other therapeutic agents to be administered for a longer period of time.

Exemplary additional active agents include bronchodilators and corticosteroids.

Suitably, bronchodilators are medicines that relax smooth muscle around the airways, increasing the caliber of the airways and improving air flow. They can reduce the symptoms of shortness of breath, wheeze and exercise limitation, resulting in an improved quality of life for people with COPD. Bronchodilators are usually administered with an inhaler or via a nebulizer.

The two major types of bronchodilators are 2 agonists and anticholinergics. β 2 agonists stimulate β 2 receptors on airway smooth muscles, causing them to relax. Exemplary β 2 agonists include albuterol, terbutaline, salmeterol and formoterol. Anticholinergics cause airway smooth muscles to relax by blocking stimulation from cholinergic nerves. Exemplary anticholinergics include ipratropium, tiotropium and oxitropium.

Corticosteroids typically act to reduce the inflammation in the airways, in theory reducing lung damage and airway narrowing caused by inflammation. Exemplary corticosteroids include prednisone, fluticasone, budesonide, mometasone, ciclesonide and beclomethasone. Corticosteroids are used in tablet or inhaled form to treat and prevent acute exacerbations of COPD.

### Administration

The active compound or pharmaceutical composition comprising the active compound may be administered to a subject by any convenient route of administration, whether systemically/peripherally or at the site of desired action, including but not limited to, oral (e.g. by ingestion); topical (including e.g. transdermal, intranasal, ocular, buccal, and sublingual); pulmonary (e.g. by inhalation or insufflation therapy using, e.g. an aerosol, e.g. through mouth or nose); rectal; vaginal; parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot, for example, subcutaneously or intramuscularly.

The subject may be a eukaryote, an animal, a vertebrate animal, a mammal, a rodent (e.g. a guinea pig, a hamster, a rat), murine (e.g. a mouse), canine (e.g. a dog), feline (e.g. a cat), equine (e.g. a horse), or a primate such as a monkey (e.g. marmoset, baboon), an ape (e.g. gorilla, chimpanzee, orangutan, gibbon) or a human.

### Method of Classifying a Lung Disease Patient for a Therapeutic Regimen

In an aspect, the disclosure relates to a method of classifying a patient having lung disease as a candidate for treatment with a therapeutic. The method includes detecting at least one marker in a sample from a patient having lung disease and comparing the level of the marker to a threshold level for the marker and using a difference in the levels to classify the patient as a candidate for therapy.

In some embodiments, the method may be for identifying a patient having lung disease as a candidate for therapy with a therapeutic such as, for example, an agent (e.g., an agonist or inducer) that can increase the amount of functional NAD(P)H:quinine oxidoreductase 1 (NQO1) (e.g., increase expression of the NQO1 gene, increase the amount of NQO1 protein, and/or increase the activity of NQO1 protein), or a combination therapy comprising the agent and another therapeutic regimen (e.g., antioxidant therapy) used to treat lung disease. In some embodiments, the agent can be a naphthoquinone. In some embodiments the agent can be a 1,2-naphthoquinone, a 1,4-naphthoquinone, or a 2,6-naphthoquinone. In some embodiments the agent can be lapachol or β-lapachone.

In some embodiments, the method comprises providing a sample from a patient having lung disease, determining the level of at least one marker in the sample, and classifying the patient as a candidate for therapy with an agent that increases the amount of functional NQO1 when the sample is determined to have an increased or decreased level relative to the threshold or baseline level of at least one of the markers.

In some embodiments, the method may be for predicting responsiveness of a patient having lung disease to therapy with an agent that can increase the amount of functional NQO1, or a combination therapy comprising the agent. The method may comprise providing a sample from a patient having lung disease (e.g., a tissue sample), determining the level of at least one marker in the sample, and classifying the patient as a candidate for therapy with an agent that can increase the amount of functional NQO1 when the sample has an increased or decreased level relative to a threshold or baseline level of at least one of the markers.

The method can be used for targeted lung disease therapy. In particular, the method is useful for therapy selection for patients having lung disease such as, for example, COPD, or emphysema, for a therapy that increases the amount of functional NQO1, such as a therapy comprising a naphthoquinone. The method can be used as companion assays for naphthoquinone therapy, when the regimen includes naphthoquinone therapy alone, or as part of combination therapy with another therapeutic, such antioxidant therapeutics as are known in the art.

In some embodiments, the assays include identifying a marker for either predicting therapy response, and for monitoring patient response to a therapeutic regimen such as naphthoquinone therapy, or both predicting and monitoring response. Suitably, assays for response prediction are run before start of therapy and patients showing levels of a marker above or below a threshold level of the marker are eligible to receive naphthoquinone therapy. For monitoring patient response, the assay is run at the initiation of therapy to establish baseline level of the marker in the sample (e.g., tissue (e.g., lung), bronchoalveolar lavage fluid, blood, serum, or plasma). The same sample is then assayed and the levels of the marker compared to the baseline. The marker level can indicate that the therapy is likely being effective and can be continued or if the patient may not be responding to therapy. The baseline level can be determined in a sample taken from the patient at the time of start of therapy.

The method uses observable differences between the level of a marker in a sample from a patient and a threshold level for the marker to classify the patient as a candidate for therapy. In some embodiments, a patient sample having a marker level below the threshold level indicates that the patient is a candidate for naphthoquinone therapy. In some embodiments, a patient sample having a marker level above the threshold level indicates that the patient is a candidate for naphthoquinone therapy. In some embodiments, a combination of marker levels (e.g., determining the level of two or more markers from a patient sample) can be used to evaluate and classify the patient is a candidate for naphthoquinone therapy. In some embodiments, a threshold level for a particular marker in a particular type of lung disease may be more indicative that the patient is a candidate for naphthoquinone therapy relative to another marker. Threshold levels can vary depending on the particular marker and can be determined by any appropriate method, such as statistical evaluation of data from one or more patient populations. For example, a threshold level can be determined using data from a pool of patients having similar or the same lung disease (e.g., COPD, emphysema, etc.).

The method measures the level of a marker to compare its level in a sample to a threshold level. The marker can be measured in a number of different ways depending on the nature of the marker, and can be determined by one of skill in the art. For example, the marker may be measured by analyzing chromosomal information (e.g., copy number, mutations (e.g., SNPs, deletions), etc.); nucleic acid based assays (e.g., hybridization, PCR, real-time PCR, qPCR, fluorescence in situ hybridization, microarrays, etc.); cell-based assays; or immunological methods or other protein assays (e.g., binding labeled antibody or protein to the expressed marker (e.g., ELISA, sandwich type assays employing capture and detection antibodies), mass spectrometry methods, or proteomic based or “protein chip” assays for the expressed marker). Suitably, the assays employ at least one detectable label such as routinely used in the art (e.g., fluorophores, dyes, radioactive nuclides, specific binding pairs, enzymes, and the like).

In some embodiments the method measures the level of a marker in a sample, wherein the marker is above, below, or at a threshold level. In some embodiments, the marker comprises a protein or nucleic acid molecule comprising a NQO1 sequence such as, for example, those disclosed herein (e.g., SEQ ID NOs 1-8). In some embodiments, the level of the NQO1 sequence in the sample is below a threshold level (e.g., a baseline level for the same patient or a pool of patients having the same lung disease, or a baseline level for healthy subjects).

In some embodiments, the marker comprises a molecule indicative of oxidative stress, such as, for example, 8-isoprostane level, protein carbonyl level, neutrophil level, macrophage level, or nuclear factor erythroid 2-related factor 2 (Nrf-2) expression level. In some embodiments the level of the marker is higher than the baseline or threshold level (e.g., 8-isoprostane level, protein carbonyl level, neutrophil level, macrophage level). In some embodiments the level of the marker is lower than the baseline or threshold level (e.g., Nrf-2 expression level). As noted above, these markers can be detected using any technique or assay known in the art such as, for example, the assays described in the Examples section.

An increased or a decreased level of the marker in a sample can be any detectable difference between the sample measurement and the threshold level (e.g., typically from ˜1% to over 100% higher or lower).

In some embodiments of the method disclosed herein, an increased level, relative to a threshold level or a baseline level, of 8-isoprostane, protein carbonyl, neutrophil, or macrophage identifies a patient as a candidate for antioxidant (e.g., naphthoquinone) therapy. In some embodiments an increased level from a sample taken from a patient after beginning therapy, relative to a baseline level of NQO1 or Nrf-2 identifies that a naphthoquinone-based therapy is effective (providing a clinical benefit). A threshold level can be established by analyzing NQO1, 8-isoprostane, protein carbonyl, neutrophil, macrophage, or Nrf-2 levels in healthy subjects, or by determining a baseline NQO1, 8-isoprostane, protein carbonyl, neutrophil, macrophage, or Nrf-2 level prior to therapy. Suitably, a differences in levels (e.g., either increased or decreased levels) of NQO1, 8-isoprostane, protein carbonyl, neutrophil, macrophage, or Nrf-2 in a sample can be any detectable difference between the sample measurement and the threshold or baseline level where the sample level of NQO1, 8-isoprostane, protein carbonyl, neutrophil, macrophage, or Nrf-2 is at least about 1% to over 100% different than the threshold or baseline level.

As discussed above, in some embodiments the method detects levels of markers that are useful in classifying a patient as a candidate for naphthoquinone therapy. Accordingly, the marker can be any biomolecule that is present and detectable in a patient having lung disease, is indicative of oxidative stress, and which can correlate to potential clinical benefit of naphthoquinone therapy. In some embodiments the markers may be NQO1, 8-isoprostane, protein carbonyl, neutrophil, macrophage, or Nrf-2, or any combination of two, three, four, five, six or seven of these markers. In some embodiments the marker is NQO1, or NQO1 in combination with one or more of 8-isoprostane, protein carbonyl, neutrophil, macrophage, or Nrf-2. In some embodiments the marker is 8-isoprostane, or 8-isoprostane in combination with one or more of NQO1, protein carbonyl, neutrophil, macrophage, or Nrf-2. In some embodiments the marker is protein carbonyl or protein carbonyl in combination with one or more of NQO1, 8-isoprostane, neutrophil, macrophage, or Nrf-2. In some embodiments the marker is neutrophil count or neutrophil count in combination with one or more of NQO1, 8-isoprostane, protein carbonyl, macrophage, or Nrf-2. In some embodiments the marker is macrophage count, or macrophage count in combination with one or more of NQO1, 8-isoprostane, protein carbonyl, neutrophil, or Nrf-2. In some embodiments the marker is Nrf-2 or Nrf-2 in combination with one or more of NQO1, 8-isoprostane, protein carbonyl, neutrophil, or macrophage.

In another aspect, the disclosure provides a method for identifying a subject as having an increased risk of developing lung disease. The method may comprise providing a sample from a subject, determining the presence or absence of a functional variant of NQO1 in the sample, and identifying the patient as having an increased risk of developing lung disease when the sample is determined as having a functional variant of NQO1. In some embodiments, the functional variant comprises Pro187Ser (rs 1800566), which has been studied as a marker in association with various cancers (see, e.g., Zhou, J Y, et al., Int J Colorectal Dis (2011) e-publication; Sameer, A S., et al., Asian Pac J Cancer Prev. (2010) 11(1):209-13; Yuan, W., et al., Breast Cancer Res Treat. (2011 January); 125(2):467-72, epub 2010 Jun. 6; Chao, C., et al., Cancer epidemiol Biomarkers Prev. (2006 May) 15(5):979-987); and Zhang, J-H., et al., World J Gastroenterol (2003) 9(7):1390-1393. In some embodiments the Pro187Ser functional variant comprises a single nucleotide polymorphism at C609T of exon 6 of an NQO1 cDNA (Kuehl, B L., et al., Br J Cancer (1995) 72: 555-561, incorporated herein by reference).

The method can be used for targeted therapy. In particular, the method is useful for identifying a subject who may have an increased risk of developing lung disease such as, for example, COPD or emphysema, and classifying the subject as a likely responder to a therapy comprising an antioxidant therapeutic. In some embodiments the antioxidant therapy comprises a naphthoquinone, such as a naphthoquinone described above.

### EXAMPLES

### Methods

Animals:

C57BL/6J mice were purchased from Jackson Laboratories (Bar Harbor, Me.) to be used as controls. A breeding colony was established at Duke University from breeding pairs of NQO1 deficient mice (backcrossed 16 generations on a C57BL/6 background) that were generously provided by Dr. Frank Gonzalez at the National Cancer Institute (Bethesda, Md.) (Radjendirane et al. J. Biol. Chem. 1998; 273:7382-7389). Male C57BL/6 or NQO1 deficient mice were used at one, two, four and six months of age. Experimental protocols were approved by the Institutional Animal Care and Use Committee at Duke University Medical Center and were performed in accordance with the standards established by the U.S. Animal Welfare Act.

Elastase Treatment:

At two months of age, WT and NQO1 deficient mice were treated with porcine pancreatic elastase (PPE, Sigma Aldrich, St. Louis Mo.) or saline by oropharyngeal aspiration. Briefly, immediately after inhalational anesthesia with 3% isoflurane (IsoFlo from Abbott Laboratories and Open-Circuit Gas Anesthesia System from Stoelting), animals were suspended by their upper incisors on a 60° incline board, and a liquid volume of PPE [25 μg/50 μL saline] or saline alone was delivered by oropharyngeal aspiration. Thirteen days post, the mice were phenotyped by flexiVent for pulmonary mechanics and lavage to collect bronchoalveolar lavage fluid (Foster et al. J Appl Physiol 2001; 90:1111-1117).

Lipopolysaccharide (LPS) Treatment:

LPS (Sigma Aldrich, St Louis Mo.) was reconstituted with sterile saline and added for a dose of 5 μg/m³. At one month of age, mice were placed in stainless steel wire cage exposure racks in a 20-L plexiglass chamber and exposed to aerosolized LPS for 2.5 hours. LPS solution was aerosolized with a 6-jet atomizer (TSI) with all output directed to the exposure chamber. Filtered air was supplied to the atomizer at 30-psi gauge pressure (Brass et al. Am. J. Resp. Cell. Mol. 2008; 39:584-590). Mice were then returned to their cages. The mice were exposed every other day for a total of three exposures and then given four weeks to recover.

N-Acetyl Cysteine (NAC) Treatment:

N-acetyl cysteine (Sigma Aldrich, St. Louis Mo.) was dissolved in the mouse water at a concentration of 2 mg/mL (March et al. Toxicol. Sci. 2006; 92:545-559). The water was changed every two days to prevent oxidation of the NAC. Mice were given NAC in their water for one week before LPS/Saline or PPE/Saline treatment and remained on the NAC water until phenotyping.

Static Compliance and Pressure-Volume Curves:

For the mice that underwent pulmonary function tests, the procedure required approximately five minutes per mouse from the point at which the mouse was administered anesthetic and prepared for surgery until the conclusion of pulmonary function measurements and euthanasia. Mice were anesthetized (60 mg/kg Nembutal) and surgically prepared with a tracheal cannula and then placed on a computer-controlled ventilator (flexiVent, SCIREQ, Montreal, Canada) at a constant tidal volume of 7.5 mL/kg and a peak expiratory end pressure of 3 cm H₂O. The animals were then given a neuromuscular blockade (0.8 mg/kg Pancuronium Bromide, Sigma-Aldrich) and given three minutes to adjust to the ventilator. Pressure-volume curves were generated by slow stepwise (ramp) inflation to total lung capacity and deflation back to forced residual capacity (Lovgren et al. Am. J. Physiol. 2006; 291:L144-156). The static compliance measurements were calculated from the pressure volume curves (Cₛₜ cmH₂O/s/mL).

Morphometric Assessment of Alveolar Development:

Alveolar surface density (ASD) was calculated as previously described (Auten et al. Am. J. Physiol. 2001; 281:L336-344). Briefly, ten digital images of parenchymal architecture from each mouse were captured at 40× magnification were chosen (omitting large vascular and bronchiolar structures) from 5 μm thick sections of paraffin embedded, formalin-fixed mouse lung that were stained with hematoxylin and eosin (H&E stain, AML Laboratories, Rosedale, Md.). Five random fields were chosen (omitting large vascular and bronchiolar structures) and images were overlaid with an 11°-11 point grid (Metamorph; Universal Imaging, West Chester, Pa.) for point (P) and intercept (I) counting of the alveolar septa. Alveolar volume density was calculated from Pₐₗᵥₑₒₗᵢ/P_{parenchyma} and alveolar surface density from 2Iₐₗᵥₑₒₗᵢ/LT, where LT was the test line length within the lung parenchyma. For mean linear intercept (MLI), a series of grid lines is laid over each photomicrograph to determine number of times those lines are intercepted by alveolar tissue where Lₘ=L/Lᵢ where L is the total length of the lines in the grid field, and Lᵢ is the total number of times those lines are intercepted (Lindsey et al. Am. J. Resp. Crit. Care 2011 Jun. 15; 183(12):1644-1652). Data from each group are expressed as mean±SE.

Residual Volume:

C57BL/6 and NQO1 deficient mice were anesthetized (60 mg/kg Nembutal), the trachea was cannulated and then the mice were placed on a computer-controlled ventilator (flexiVent, SCIREQ, Montreal, Canada). The mice were immediately given a side breath and then a pressure volume curve was obtained. The ventilator was then supplied with 100% oxygen. After 10 minutes, the cannula was clamped (during normal pulmonary perfusion) and residual oxygen was absorbed thus degassing the lung. The mouse was removed from the ventilator and the lung was carefully excised and tied to a weight. Using Archimedes' principle, the lung and weight were placed in a beaker with water and weighed ensuring the lung remained submerged in the water. By subtracting the weight of the beaker, water, weight and cannula from this weight, the excised lung gas volume was determined.

Gross Anatomy:

Mice were euthanized at each age and the lungs were filled to total lung capacity to 25 cm of H₂O of pressure with 10% formalin, sutured at the trachea and removed. After forty-eight hours, the hearts were removed from the lung and the lungs were photographed.

Histology:

Mice were euthanized and a catheter was placed in the trachea. The lungs were filled to total lung capacity (25 cm of H₂O of pressure) with 10% formalin and allowed to sit for thirty minutes. The left lung was then sutured at the bronchus, removed, and placed in a 15 mL conical with 10% formalin. After 48 hours, the lungs were switched to 70% EtOH, embedded, cut and stained for H&E staining.

Bronchoalveolar Lavage Fluid (BALF):

Immediately after pulmonary function measurements, mice were overdosed with Nembutal (100 mg/kg) to euthanize. The chest was opened, the trachea was exposed, and bronchoalveolar lavage (BAL) was performed by intubating the mouse trachea with PE-90 tubing and instilling saline until the lung reached total lung capacity to 25 cm H₂O pressure. This was repeated three times. The total volume returned was the lavage return volume. The left lung was inflated through the trachea with 10% formalin, fixed in 10% formalin, stored at 4° C. for 24 h, and paraffin embedded and sectioned for further study. Cells from the BALF were isolated using centrifugation (1500 rpm, 15 minutes) and the supernatant was stored at −80° C. for assessment of 8-isoprostane. Cells were resuspended in Hank's balanced salt solution (1 mL) and counted via Millipore Scepter (Millipore). Cell differential was determined from an aliquot of the cell suspension (100 uL) by centrifugation on a slide (Cytospin 4: Shandon, Pittsburgh, Pa.). Differential cell counts were expressed as number of cells/mL, means±SEM for each group of animals (Li et al. J. Leukocyte Biol. 2009; 85:124-131).

8-Isoprostane:

8-isoprostanes were measured in both the BALF supernatant and the cell supernatant using purification columns and an EIA assay kit from Cayman Chemical Company (Ann Harbor, Mich.). Briefly, samples were diluted 1:2 with column buffer and applied to the purification columns. The sample passed entirely through the column. The column was then washed with column buffer and ultrapure water and the washes were discarded. 5 mL of elution solution was added to the column and allowed to pass through in order to elute the 8-isoprostane. The solution passed through the column was then collected in a 5 mL tube and the elution solution was evaporated to dryness using a stream of dry nitrogen gas in order to remove all quantities of organic solvent. The purified samples were then reconstituted with saline and used for the EIA kit (Cayman Chemical, Ann Harbor, Mich., USA) following the manufacturer's protocol. Samples, standards, buffer, bound 8-isoprostane AChE Tracer, and antiserum were added the plate and incubated at 40 degrees for 18 hours. The plate was then washed five times with wash buffer and the Ellman's reagent (substrate for AChE tracer) was added. After 90 minutes, the plate was read on a plate reader at a wavelength of 405 nm. The 8-isoprostane concentrations was calculated by plotting the percent ratio of standard bound/maximum bound for each of the standards using linear and log axes and performing a 4-parameter logistic fit.

Protein Carbonyl:

Protein Carbonyls were measured in both the BALF supernatant and the cell supernatant using an OxiSelect Protein Carbonyl ELISA kit following the manufacturer's protocol (Cell Biolabs, Inc., San Diego, Calif., USA). Briefly, BSA standards or samples were absorbed onto a 96-well plate for 2 hours at 37° C. The protein carbonyls present in the sample or standard were derivatized to DNA hydrazone and probed with an anti-DNP antibody, followed by an HRP conjugated secondary antibody. The plate was then read at 405 nm by a plate reader. The protein carbonyl content in the sample was determined by comparing with a standard curve that was prepared from predetermined reduced and oxidized BSA standards.

Glutathione:

Glutathione (GSH) concentrations were measured in the BALF supernatant using a Glutathione Assay Kit (Cayman Chemical, Ann Harbor, Mich., USA) following the manufacturer's protocol. Briefly, samples and standards were absorbed on a 96-well plate along with the assay cocktail provided (MES Buffer, Cofactor Mixture, Enyzme Mixture, DTNB and water) for 25 minutes. The plate was then read at 405 nm by a plate reader and the concentrations were determined by comparing with a standard curve that was prepared from predetermined concentrations.

MAC220/β-Lapachone/Dicumarol:

Primary alveolar macrophages were collected by bronchoalveolar lavage (BAL) with 10 ml of 0.9% NaCl containing 0.5 mM EDTA. Cells were allowed to attach to the bottom of 24-well cell culture plates for 4 hours in RPMI1640 with 10% FBS, penicillin (100 U/ml) and streptomycin (100 μg/ml) and then treated with 20 ng/ml of LPS or 1 μM of PMA for another 2 hours. Supernatants were collected for detecting oxidant products. Murine alveolar macrophage cell line (MH-S cell) purchased from ATCC (Manassas, Va.) was used for in vitro NQO1 blocking and inducing assays. Cells were cultured in RPMI1640 medium supplemented with 10% heat-inactivated fetal bovine serum, 2 mM L-glutamine, penicillin (100 U/ml), streptomycin (100 μg/ml), and 10 mM HEPES. To determine the role of NQO1 in macrophage response to LPS or PMA, cells at 80-90% confluence were pretreated with 10 μM of dicumerol for 1 hour, 100 nM of MAC220 or 5-10 μM of β-lapachone for 4 hours, and then were challenged by 20 ng/ml of LPS or 1 μM of PMA. Cell culture supernatants were collected after 2 hour incubation. Same volume of DMSO and PBS were used as vehicle control.

Statistics:

All data are expressed as mean±SEM. Two-way ANOVA for comparisons among multiple groups was performed using Graphpad Prism 5.0. Student-t test was used for individual comparisons between groups. Significance was defined as two-tailed P value of less than 0.05.

### Example 1

### Pulmonary Effect of NQO1 Deficiency in Mice

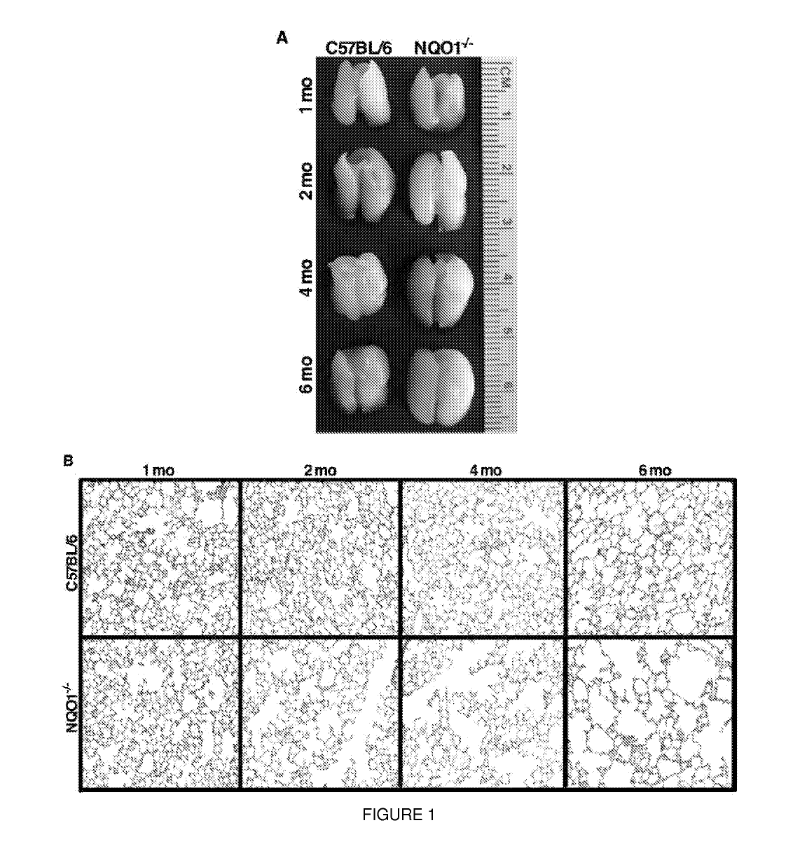

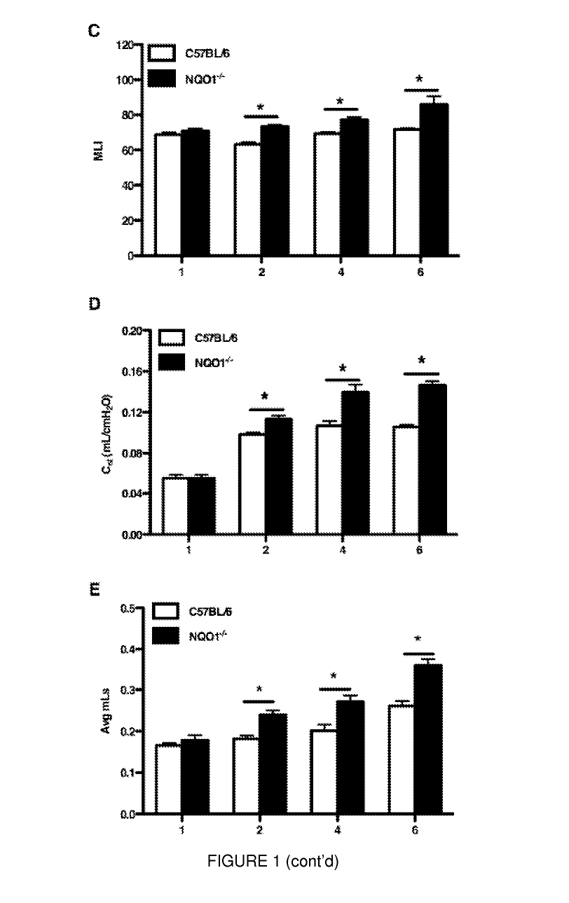

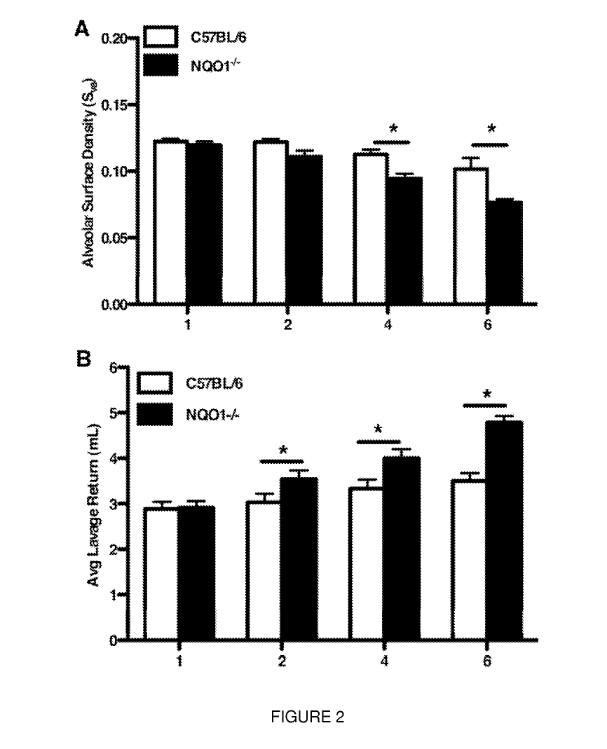

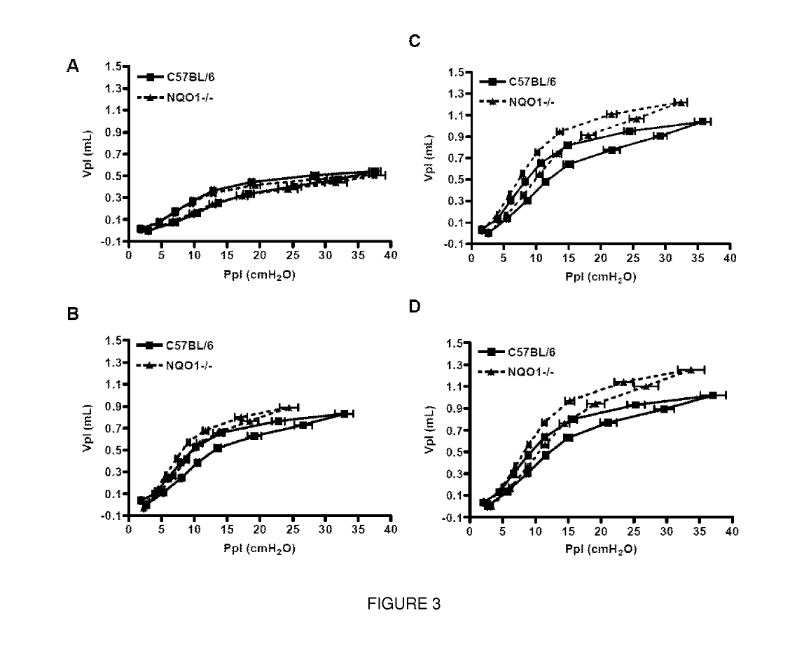

Naive NQO1 deficient mice appear to have increased lung size on necropsy as compared to naive C57BL/6 mice (FIG. 1A). This suggests a baseline difference in alveolar volume. To confirm this observation, the lung histology of NQO1 deficient and C57BL/6 mice at ages one, two, four and six months were compared (FIG. 1B). Beginning at two months of age, NQO1 deficient mice showed increased lavage return volumes that augmented with aging (FIG. 2B). NQO1 deficient mice developed progressive airspace enlargement with age, when compared to C57BL/6 mice. This alveolar enlargement was quantified by evaluating the alveolar surface density (ASD). As shown in FIG. 2A, lung histology shows decreased alveolar surface density in the NQO1−/− mice beginning at two months of age with decreased density with age consistent with the development of premature emphysema. (The alveolar surface density measurements were performed on 10 mice per group with 2 repeats.) Mean line intercepts (MLI) for NQO1 deficient and WT mice were also determined. NQO1 deficient mice, starting at two months, developed an increase in MLI, which appeared to be progressive over later time points (FIG. 1C). There was no appreciable difference in the MLI and ASD for WT mice over the same time points. As the lungs of the NQO1 deficient mice showed apparent alveolar enlargement based on histology, detailed lung function measurements were performed to confirm these findings (FIG. 1C, 1D, 2A). Enhanced lung volumes and increased pulmonary compliance are characteristic features of human emphysema (Shapiro S D, Curr. Opin. Cell Biol. 1998; 10:602-608). To determine whether this murine model recapitulated these important clinical features of emphysema, the lung volumes and compliance of C57BL/6 and NQO1 deficient mice were compared at one, two, four and six months of age. Beginning at two months of age (FIG. 1D), NQO1 deficient mice had increased lung compliance compared to C57BL/6 mice. Furthermore, pressure-volume curves (FIG. 3A-3D) from naive NQO1 deficient mice demonstrated a steady increase in lung compliance with age. Residual lung volumes (FIG. 1E) of degassed lungs of both NQO1 deficient and C57BL/6 mice were also compared; the same pattern of increased lung volumes in the NQO1 deficient mice with age was observed. These findings demonstrate that NQO1 deficient mice spontaneously develop emphysema with aging. These observations support that NQO1 protects against the development of spontaneous emphysema.

### Example 2

### Effect of Elastase Treatment in NQO1 Deficient Mice

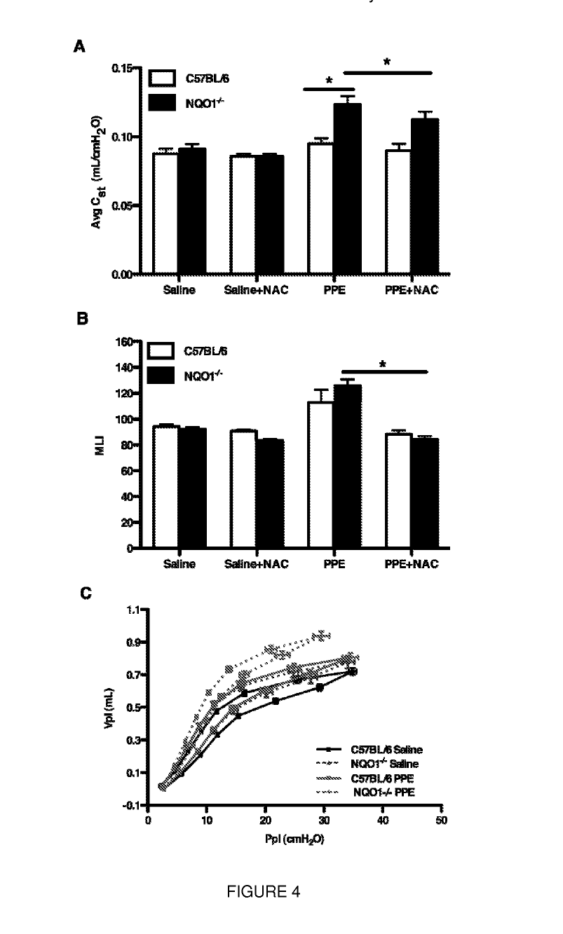

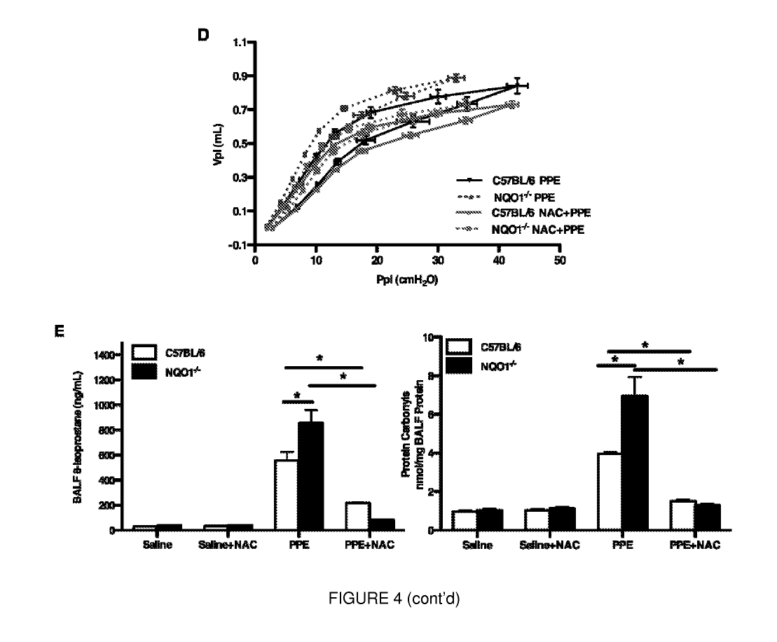

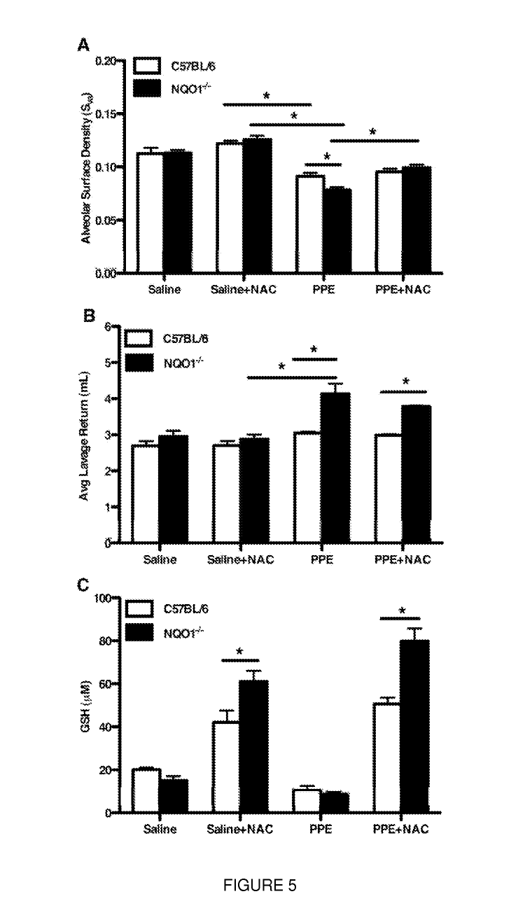

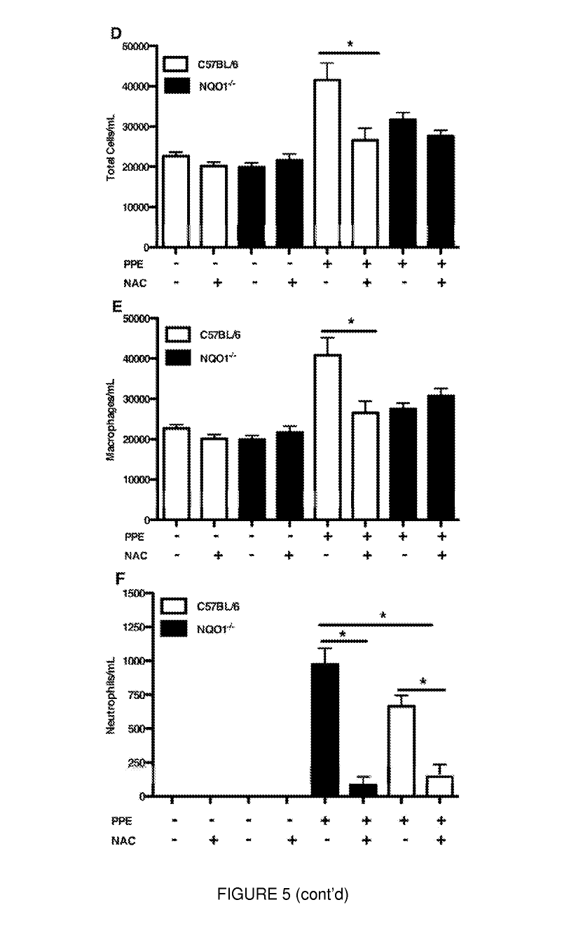

The effect of NQO1 on the development of emphysema in known experimental models was also evaluated. First, the role of enhanced oxidant burden in the development of enhanced emphysema in NQO1 deficient mice was determined. Intratracheal administration of elastase has been previously demonstrated to result in the development of emphysema in murine models (Borzone et al. Am J Physiol Regul Integr Comp Physiol 2009; 296:R1113-1123; Hantos et al. J Appl Physiol 2008; 105:1864-1872). At four weeks of age, NQO1 deficient and C57BL/6 mice were given porcine pancreatic elastase (PPE) or saline by oropharyngeal aspiration and phenotyped thirteen days post treatment. Elastase administration caused a significant increase in lung compliance in the NQO1 deficient mice, but not in the C57BL/6 mice (FIG. 4A). This finding was also confirmed with analysis of pressure volume curves, mean line intercepts and alveolar surface density (FIGS. 4B, 4C, 4D and 5A). We hypothesized that enhanced emphysema was due to increased oxidant stress in the NQO1 deficient mice as compared to wild type. We determined that after elastase instillation NQO1 deficient mice had enhanced 8-isoprostane and protein carbonyls over C57BL/6 mice (FIG. 4E). To determine if the lack of NQO1 antioxidant enzymatic activity was responsible for the emphysema phenotype, we treated NQO1 deficient and C57BL/6 mice with an exogenous antioxidant, N-acetyl cysteine. NAC is a precursor of glutathione molecules and has oxygen radical-scavenging properties (Gillissen et al. Respiratory medicine 1998; 92:609-623). NAC was provided in the water of NQO1 deficient and C57BL/6 mice at three weeks of age and then followed by intratracheal elastase administration at four weeks. Treatment with oral NAC partially attenuated lung compliance and pressure-volume measurements in the NQO1 deficient mice (FIG. 4D) compared to the elastase only treated groups. Similar, yet more striking results were found by comparison of the surface alveolar density and mean line intercepts between PPE and PPE+NAC groups with a complete abrogation of the elastase effect (FIGS. 4B and 5A). Lavage return volumes were higher in the NQO1 deficient mice treated with PPE compared to the C57BL/6 mice (FIG. 5B). The reduction in emphysematous changes in NQO1 and WT mice that were given NAC prior to elastase challenge was associated with a reduction in 8-isoprostane levels and protein carbonyls (FIG. 4E). Glutathione measurements in the BAL showed increased concentrations in the NQO1 deficient mice treated with NAC (FIG. 5C). Total and differential cell counts showed an increase in both total cells and macrophages in the C57BL/6 mice treated with PPE (FIGS. 5D and 5E). Both the NQO1 deficient mice and the C57BL/6 mice treated with PPE showed a significant increase in neutrophils compared to the PPE+NAC group (FIG. 5F). These observations support that NQO1 has a critical role in protecting against elastase-induced emphysema and suggest that this effect is primarily by reduction of oxidant burden in the lung.

### Example 3

### Oxidant Stress in NQO1 Deficient Mice

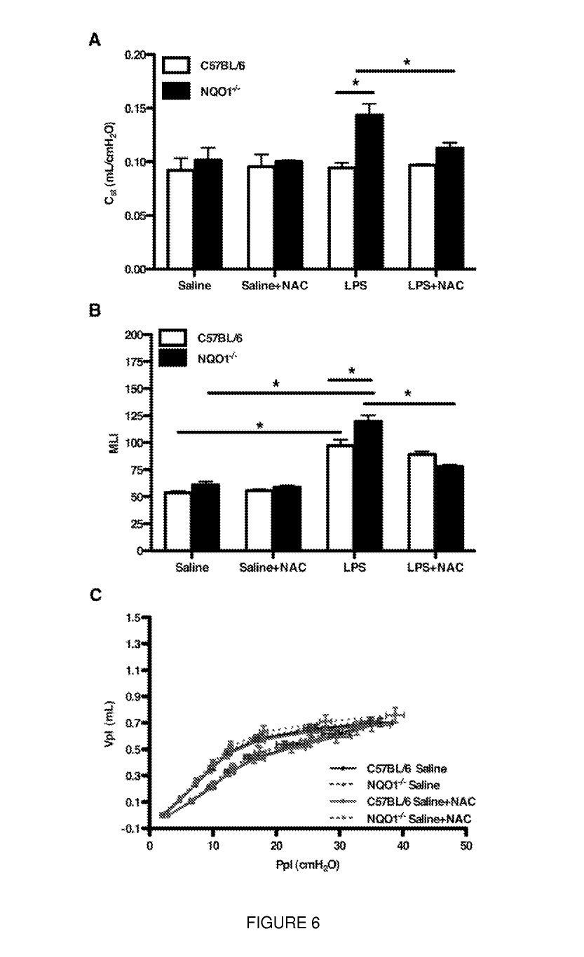

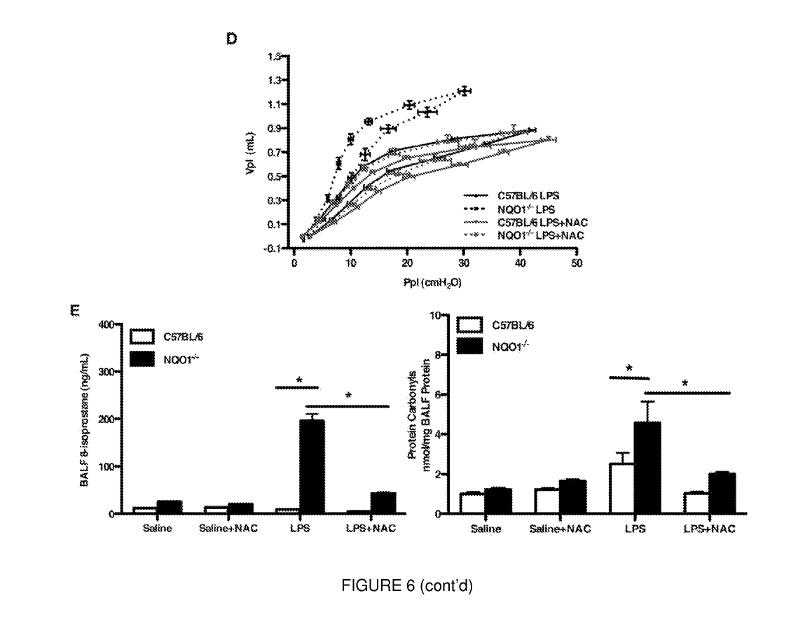

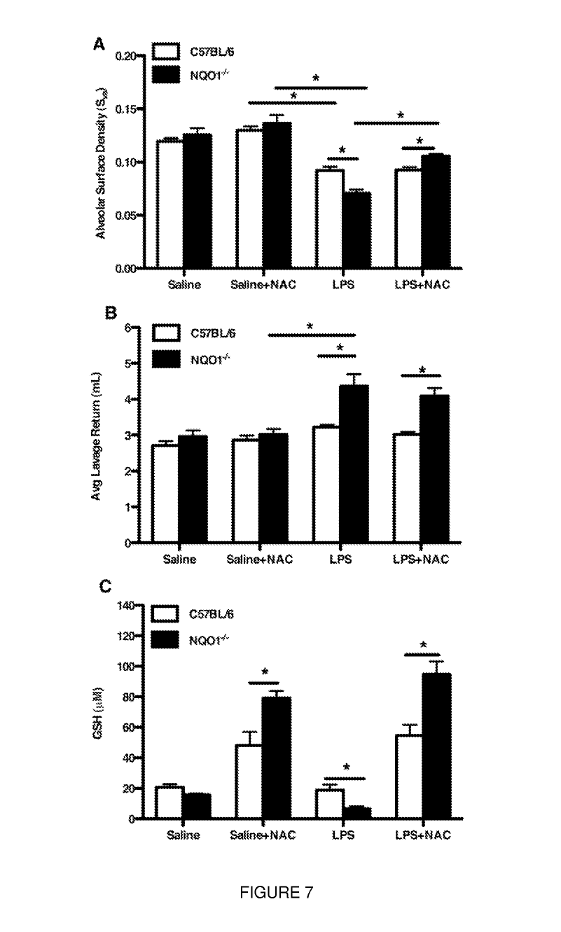

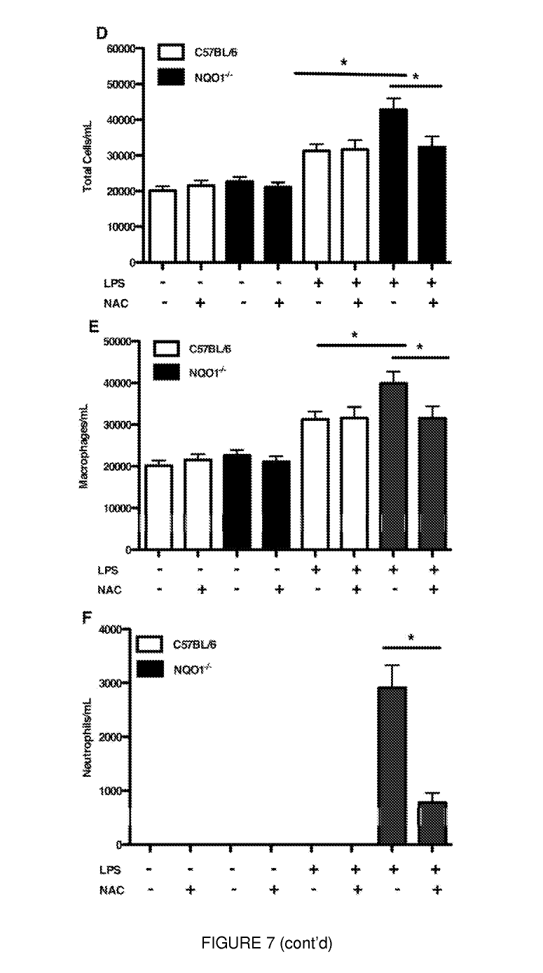

Lipopolysaccharide (LPS) may induce emphysema, and may induce oxidant stress. The effect of sub-chronic LPS exposure was therefore evaluated in NQO1 deficient mice. To determine the in vivo biological consequence of enhanced LPS-induced oxidant stress, a modified model of LPS-induced murine emphysema was developed. At three weeks of age, NQO1 deficient and C57BL/6 mice were treated with either standard water or water with the addition of NAC. At four weeks of age, the mice underwent three acute exposures (every other day for 2.5 hours per day for three exposures) to aerosolized LPS and were phenotyped four weeks after the exposures. LPS treated NQO1 deficient mice had increased static compliance when compared to LPS exposed C57BL/6 mice (FIG. 6A). As demonstrated with elastase exposure, the addition of NAC attenuated the effect of chronic LPS on the enhanced static compliance in NQO1 deficient mice. Analysis of mean line intercepts, alveolar surface density and pressure volume curves demonstrated similar findings to the compliance data (FIGS. 6B, 6C, 6D and 7A). Average lavage return volumes were increased in the NQO1 deficient mice treated with LPS compared to the C57BL/6 mice (FIG. 7B). Glutathione measurements in the BAL showed increased concentrations in the NQO1 deficient mice when treated with NAC (FIG. 7C). Analysis of the bronchoalveolar lavage fluid (BALF) revealed a significant increase in the number of total inflammatory cells (FIG. 7D), macrophages (FIG. 7E) and polymorphonuclear leukocytes (PMNs) (FIG. 7F) in the NQO1 deficient mice exposed to LPS. Among the inflammatory cell population, macrophages were the predominant cell type, constituting nearly 100% of the total cells in the C57BL/6 mice and as much as 91-100% of the NQO1 deficient mice in the BALF of mice exposed to LPS (FIG. 7E). Polymorphonuclear leukocytes constituted 1-4% of the LPS+NAC treated NQO1 deficient mice and 5-9% of the LPS only treated NQO1 deficient mice (FIG. 7F). 8-isoprostane concentrations and protein carbonyls (FIG. 6E) measured in the BALF from LPS treated NQO1 deficient mice showed a significant increase when compared to both LPS treated C57BL/6 mice and LPS+NAC NQO1 deficient mice. These data support that NQO1 is protective in the development of emphysema after LPS exposure and support that NQO1 contributes to regulation of oxidant stress.

### Example 4

### Macrophage Derived Oxidant Stress in NQO1 Deficient Mice

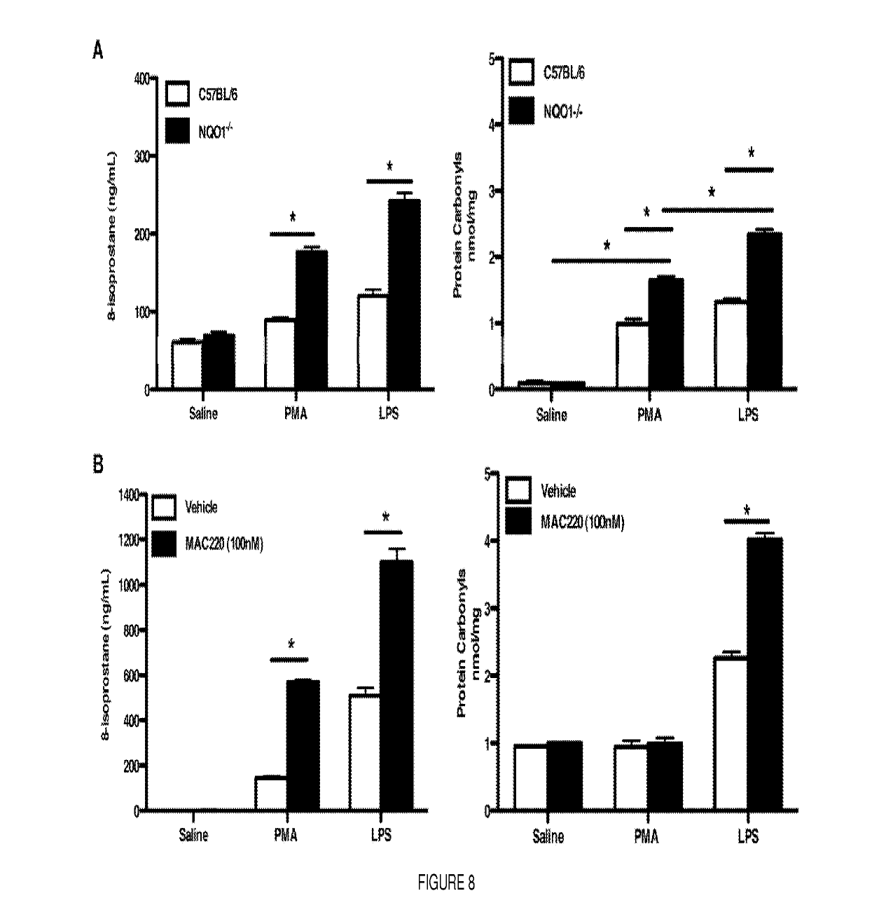

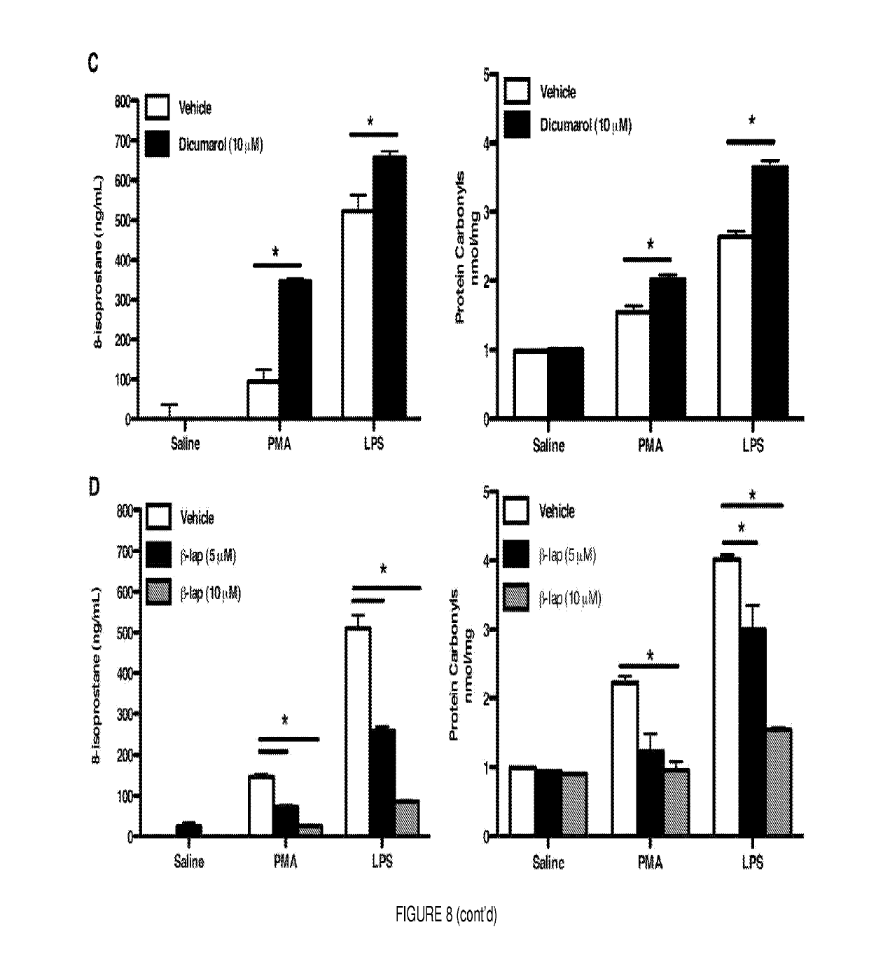

8-isoprostane and protein carbonyl levels, as markers of oxidant stress, were evaluated in NQO1 deficient mice. Given that macrophages were the dominant cell type in the BALF and have been previously implicated in the development of emphysema (26), experiments focused on macrophage-derived oxidant stress. Alveolar macrophages from NQO1 deficient mice and C57BL/6 mice (age 1 month) were obtained from bronchoalveolar lavage. The macrophages were then cultured for 12 hours and exposed to either saline, lipopolysaccharide (LPS) or phorbol myristate acetate (PMA) for an additional 2 hours. The supernatants were collected and measured for 8-isoprostanes and protein carbonyls. NQO1 deficient alveolar macrophages exposed to LPS and PMA showed an increase in 8-isoprostane and protein carbonyls compared to similarly exposed C57BL/6 alveolar macrophages (FIG. 8A). To examine whether modulating the activity of NQO1 in macrophages would alter oxidative stress, a macrophage cell line was treated with known NQO1 inducer and inhibitors. β-lapachone is bioactivated by NQO1 and is a known inducer of NQO1 (Blanco et al. Cancer Res. 2010; 70:3896-3904). Dicumarol is a recognized nonspecific inhibitor of NQO1, however it is known to have many ancillary effects other than inhibition effects of NQO1, with over a dozen enzymes that have been reported to be inhibited by dicumarol mainly in the dehydrogenase and reductase categories (Ross et al. Cancer Metast. Rev. 1993; 12:83-101). MAC220 is a potent and specific mechanism-based inhibitor of NQO1 (Dehn et al. Mol. Cancer Ther. 2006; 5:1702-1709). Using the mouse alveolar macrophage cell line (MHS cells), cells were pretreated with the NQO1 inhibitor (MAC220 or dicumarol or a NQO1 inducer (β-lapachone) followed by exposure to saline, lipopolysaccharide (LPS), or phorbol myristate acetate (PMA) for 2 hours. The supernatants were collected and the levels of oxidative stress were quantified. Macrophages treated with the MAC220 inhibitor exhibited significantly higher oxidative stress level in both the PMA exposed and LPS exposed compared to the uninhibited cells (FIG. 8B). Cells pretreated with dicumarol showed significant increased 8-isoprostane and protein carbonyls in the PMA treated and LPS treated groups (FIG. 8C). Pre-treatment of cells with the NQO1 inducer, β-lapachone, demonstrated a dose response decrease in oxidative stress levels in both the PMA and LPS treated groups (FIG. 8D). These results demonstrated that alveolar macrophages from NQO1 deficient mice have enhanced oxidant stress in response to LPS. Using an alveolar macrophage cell line, it was demonstrated that the level of oxidative stress could be modulated by altering the activity of NQO1. These data demonstrate a potent antioxidant role of NQO1 in macrophages.

### Example 5

### Increased MMP12 Expression in NQO1 Deficient Mice

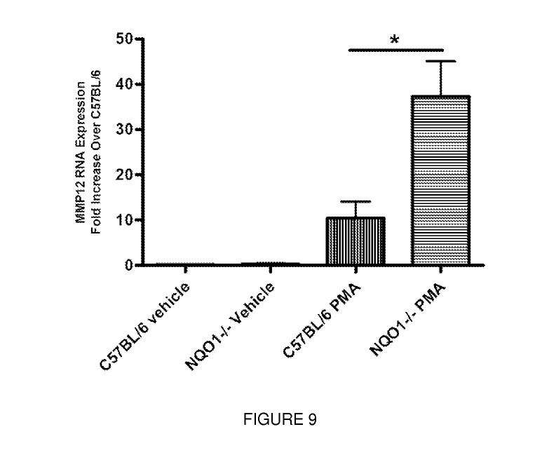

Results are illustrated in FIG. 9. Macrophages derived from NQO1−/− mice show a significant increase in MMP12 RNA expression after exposure to PMA when compared to C57BL/6 mice. Data are mean±standard errors. *significant difference (p<0.05) as determined by Student T-test (n=4). Thus, NQO1 may regulate macrophage derived MMP12 expression.

### Example 6

### Lung Function in Subjects with NQO1 Variants

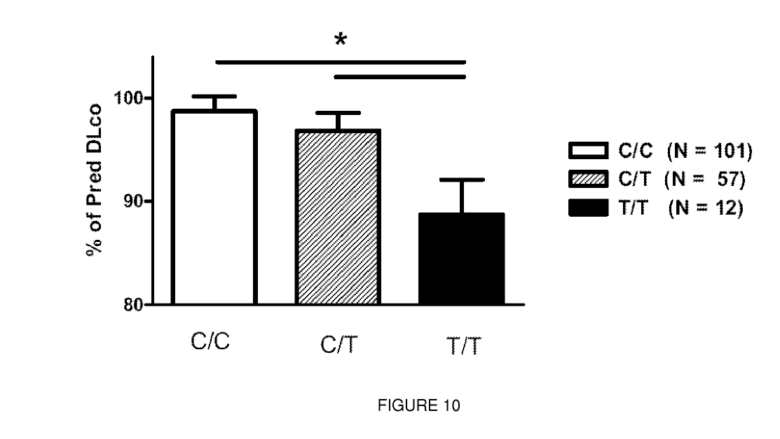

Lung function was characterized in 170 healthy 18-35 year-old human subjects. The subjects had no pre-existing medical conditions and had normal lung function (FEV1 and FVC). These healthy subjects were genotyped for a common functional variant of NQO1 (NQO1 Pro187Ser: rs 1800566) which is typically present in approximately 7% of the general population. Individuals homozygous for the minor variant of NQO1 had approximately a 10% drop in their DLCO (unpaired, two-tailed T-test, P<0.05). (See FIG. 10). This observation supports that loss of NQO1 function is associated with impaired gas exchange in the lung in asymptomatic healthy subjects. This finding suggests a dominant role of NQO1 in protecting the human lung from premature alterations in lung function, and provides support that identifying a subject (e.g., genotyping) having of functional NQO1 variant can provide for early risk assessment for eventual development of a lung disease as well as early therapeutic intervention with a tailored therapy (e.g., antioxidant therapy).

Although the disclosure above has been described in terms of various aspects and specific embodiments, it is not so limited. A variety of suitable alterations and modifications for operation under specific conditions will be apparent to those skilled in the art. It is therefore intended that the following claims be interpreted as covering all such alterations and modifications as fall within the true spirit and scope of the invention.

All patents, publications and references cited herein are hereby fully incorporated by reference. In case of conflict between the present disclosure and incorporated patents, publications and references, the present disclosure should control.

1. A method of treating a lung disease in a subject, comprising administering to the subject an effective amount of a naphthoquinone and a bronchodilator, wherein the naphthoquinone and the bronchodilator are administered via inhalation.

2. The method of claim 1, wherein the lung disease is selected from chronic obstructive pulmonary disease (COPD), emphysema, chronic bronchitis, asthma, reactive airway disease and pulmonary fibrosis.

3. The method of claim 2, wherein the lung disease is COPD.

4. The method of claim 1, wherein the naphthoquinone is a 1,2-naphthoquinone.

5. The method of claim 4, wherein the 1,2-naphthoquinone is selected from the group consisting of β-lapachone, tanshinone and cryptotanshinone.

6. The method of claim 1, wherein the naphthoquinone and bronchodilator are administered with at least one additional therapeutic agent.

7. The method of claim 6, wherein the additional therapeutic agent is a corticosteroid.

8. The method of claim 1, wherein the bronchodilator is selected from an anti-cholinergic and a beta-agonist.

9.-19. (canceled)

20. A method of predicting responsiveness of a subject with a lung disease to treatment with a naphthoquinone, comprising:
a) detecting the level or activity of NQO1 in a sample obtained from the subject; and
b) comparing the level or activity of NQO1 in the sample to a standard level, wherein an altered level or activity of NQO1 indicates that the subject is responsive to treatment with a naphthoquinone.

21. A method of identifying a subject with a lung disease as a candidate for treatment with a naphthoquinone, comprising:
a) detecting the level or activity of NQO1 in a sample obtained from the subject; and
b) comparing the level or activity of NQO1 in the sample to a standard level, wherein a difference in the level or activity of NQO1 indicates that the subject is a candidate for treatment with a naphthoquinone.

22. A method for identifying a subject as having an increased risk of developing lung disease, comprising:
a) providing a sample from a subject;
b) determining the presence or absence of a functional variant of NQO1 in the sample; and
c) identifying the patient as having an increased risk of developing lung disease when a functional variant of NQO1 is present in the sample.

23. The method of claim 22, wherein the functional variant of NQO1 is Pro187Ser.
